(19) United States
(12) Patent Application Publication
(10) Pub. No.: US 2011/0201781 A1
(43) Pub. Date: Aug. 18, 2011

(54) CYSTEINE LABELING SYSTEM AND METHOD OF USE THEREOF

(76) Inventors: Jianghong Rao, Sunnyvale, CA (US); Hongjun Ren, Hangzhou (CN)

(21) Appl. No.: 12/998,105

(22) PCT Filed: Sep. 16, 2009
(86) PCT No.: PCT/US2009/057224

§ 371 Date: Mar. 16, 2011

Related U.S. Application Data
(60) Provisional application No. 61/097,349, filed on Sep. 16, 2008.

(51) Int. Cl. C07K 1/107; C07C 63/04; C07K 14/00; C07K 2/00

(52) U.S. Cl. 530/345; 562/426; 530/408

(57) ABSTRACT

A method of use of a cysteine labeling system includes: providing a 2-cyano benzothial core with a covalently-linked biomolecule X in a reaction environment; and reacting the 2-cyano benzothial core to an N-terminal cystenine.

### CROSS-REFERENCE TO RELATED APPLICATION(S)

This application claims the benefit of U.S. Provisional patent application Ser. No. 61/097,349 filed Sep. 16, 2008, and the subject matter thereof is incorporated herein by reference thereto.

### TECHNICAL FIELD

The present invention relates generally to bioconjugation chemistries, and more particularly a system for conjugating biomolecules to cysteine-terminated molecules.

### BACKGROUND ART

A number of conjugation chemistries have been developed to conjugate biomolecules to each other or to organic molecules. Bioconjugations are important for labeling biomolecules with probes such as fluorescent dyes or proteins. Labeled biomolecules are used for biochemical assaying in biopharmaceutical and basic research, for imaging of biochemical reactions inside living subjects, and for the formulation of in vitro diagnostics tests among many other examples.

Some existing conjugation methods require conjugation conditions that are not compatible with biomolecules while other may affect the function of a given biomolecule. For example, non-specific conjugation of biotin or streptavidin to firefly luciferase may drastically reduce the activity of this enzyme. There is an important need for a conjugation chemistry that specifically binds to a given molecule that is unique to the biomolecule of interest.

Thus, a need still remains for bioconjugation techniques that are specific to a given molecule and that bind to it without significantly altering the function of a host biomolecule. In view of the expanding needs to understand biological processes and disease, it is increasingly critical that answers be found to these problems. In view of the ever-increasing commercial competitive pressures, along with growing consumer expectations and the diminishing opportunities for meaningful product differentiation in the marketplace, it is critical that answers be found for these problems. Additionally, the need to reduce costs, improve efficiencies and performance, and meet competitive pressures adds an even greater urgency to the critical necessity for finding answers to these problems.

Solutions to these problems have been long sought but prior developments have not taught or suggested any solutions and, thus, solutions to these problems have long eluded those skilled in the art.

### DISCLOSURE OF THE INVENTION

The present invention provides a method of use of a cysteine labeling system including: providing a 2-cyano benzothial core with a covalently-linked biomolecule X in a reaction environment; and reacting the 2-cyano benzothial core to an N-terminal cystenine.

In addition, the present invention provides a cysteine labeling system , including: a 2-cyano benzothial core with a covalently-linked biomolecule X in a reaction environment; and an N-terminal cystenine for forming a bond to the 2-cyano benzothial core.

Certain embodiments of the invention have other steps or elements in addition to or in place of those mentioned above. The steps or element will become apparent to those skilled in the art from a reading of the following detailed description when taken with reference to the accompanying drawings.

### BRIEF DESCRIPTION OF THE DRAWINGS

FIG. 1 is a cysteine labeling system in an embodiment of the present invention.

FIG. 2 is a schematic diagram describing a cyclization reaction between the cysteine labeling system and an N-terminal cystenine.

FIG. 3 is a schematic diagram of a conjugation product resulting from the conjugation of the cysteine labeling system of FIG. 1 conjugated to a peptide containing the N-terminal cysteine.

FIG. 4 is a schematic diagram describing a cyclization reaction between a protein with an N-terminal cysteine and a cysteine labeling system consisting of the 2-cyano benzothiazol core with a fluorescent dye covalently linked at 6 position of the 2-cyano benzothiazol core.

FIG. 5 is a schematic representation of a labeling strategy for labeling a HeLa cell transfected with a cell cyan fluorescent protein (CFP) having a tobacco etch virus protease substrate fused at its N-terminus.

FIG. 6 is a flow chart of a method of use of the activatable bioluminescent probe system 100 in a further embodiment of the present invention.

### BEST MODE FOR CARRYING OUT THE INVENTION

The following embodiments are described in sufficient detail to enable those skilled in the art to make and use the invention. It is to be understood that other embodiments would be evident based on the present disclosure, and that system, process, or mechanical changes may be made without departing from the scope of the present invention.

In the following description, numerous specific details are given to provide a thorough understanding of the invention. However, it will be apparent that the invention may be practiced without these specific details. In order to avoid obscuring the present invention, some well-known circuits, system configurations, and process steps are not disclosed in detail.

The drawings showing embodiments of the system are semi-diagrammatic and not to scale and, particularly, some of the dimensions are for the clarity of presentation and are shown exaggerated in the drawing FIGs. Similarly, although the views in the drawings for ease of description generally show similar orientations, this depiction in the FIGs. is arbitrary for the most part. Generally, the invention can be operated in any orientation.

The same numbers are used in all the drawing FIGs. to relate to the same elements. The embodiments have been numbered first embodiment, second embodiment, etc. as a matter of descriptive convenience and are not intended to have any other significance or provide limitations for the present invention.

Embodiments of the present disclosure will employ, unless otherwise indicated, techniques of medicine, organic chemistry, biochemistry, molecular biology, pharmacology, and the like, which are within the skill of the art. Such techniques are explained fully in the literature.

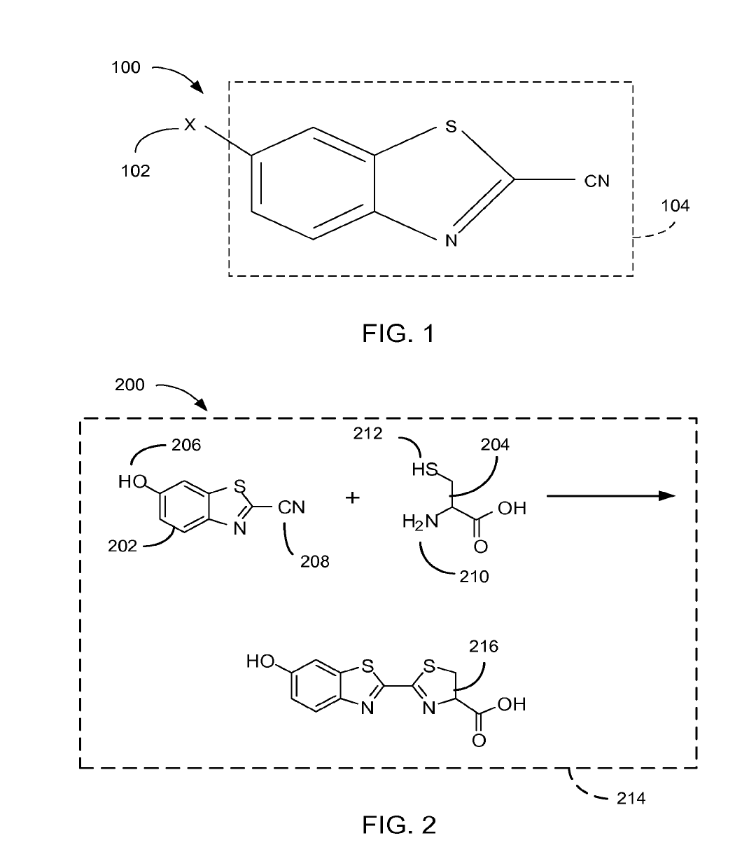

Referring now to FIG. 1, therein is shown a cysteine labeling system 100 in an embodiment of the present invention. The cysteine labeling system 100 enables conjugation of a covalently-linked biomolecule X 102 to any N-terminal cysteine-terminated biomolecule such as a peptide or a protein. In this embodiment of the invention, the cysteine labeling system 100 has a 2-cyano benzothiazol core 104 (i.e., a CBT core). The covalently-linked biomolecule X 102 is located at the 6 position of the 2-cyano benzothiazol core 104.

The covalently-linked biomolecule X 102 may be a functional group, a fluorescent dye, a bioluminescent protein, a biotin, a non-fluorescent labeling group such as a radioisotope, or a magnetic resonance imaging probe such as gadolinium. The fluorescent dye may be fluorescein isothiocynate (FITC), rodhamine, cyanine dyes, BODIPY, and ALEXA dyes among many other possible examples.

It has been discovered that the cysteine labeling system 100 specifically reacts with a peptide or protein having an N-terminal cysteine, and does not react with other cysteines that are not at an N-terminal.

Referring now to FIG. 2, therein is shown a schematic diagram describing a cyclization reaction 200 between the cysteine labeling system 202 and an N-terminal cystenine 204. In this embodiment of the present invention, the covalently-linked biomolecule X 102 of FIG. 1 is a hydroxyl group 206. In the cyclization reaction 200, a cyano group 208 in the cysteine labeling system 202 reacts an amine group 210 and a thiol group 212 in the N-terminal cystenine 204. The cyclization reaction 200 takes place in a reaction environment 214 at nearly physiological conditions. The reaction product 216 is a benzothiazolyl-thiazoline-4 carboxylic acid molecule.

The term “reaction environment” as used herein is defined as any in vitro, in vivo, or ex vivo environment in which the cysteine labeling system 302 is implemented. The reaction environment 314 may be a buffer solution; a biological liquid such as urine or blood, or a host among many other examples.

The term “host” as used herein refers to any living organism including mammals, humans, other living organisms, and samples such as cell or tissue taken from a living organism. A living organism can be as simple as a single eukaryotic cell or as complex as a human.

It has been discovered that the cyclization reaction 200 can be performed under physiological conditions, making compatible for labeling any peptide or protein with an N-terminal cysteine while minimizing disruption of the peptide or protein structure and conformation during the labeling process. Further, since the cysteine labeling system 202 is a small label, it is less likely to disrupt the conformation of a protein.

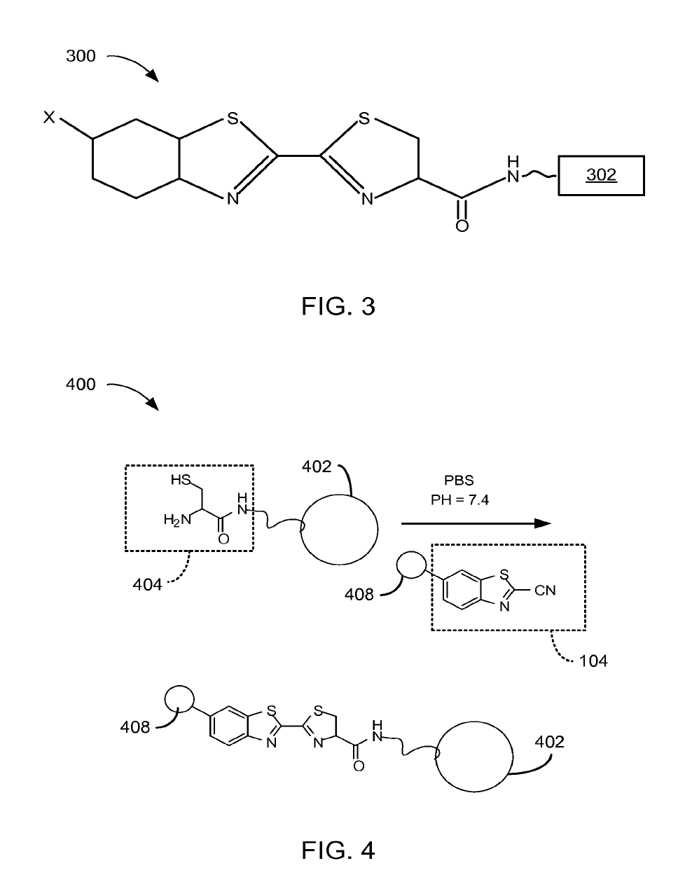

Referring now to FIG. 3, therein is shown a schematic diagram of a conjugation product 300 resulting from the conjugation of the cysteine labeling system 100 of FIG. 1 conjugated to a peptide 302 containing the N-terminal cysteine. The conjugation of the cysteine labeling system 100 to the peptide 302 is site-specific to its N-terminal cysteine residue.

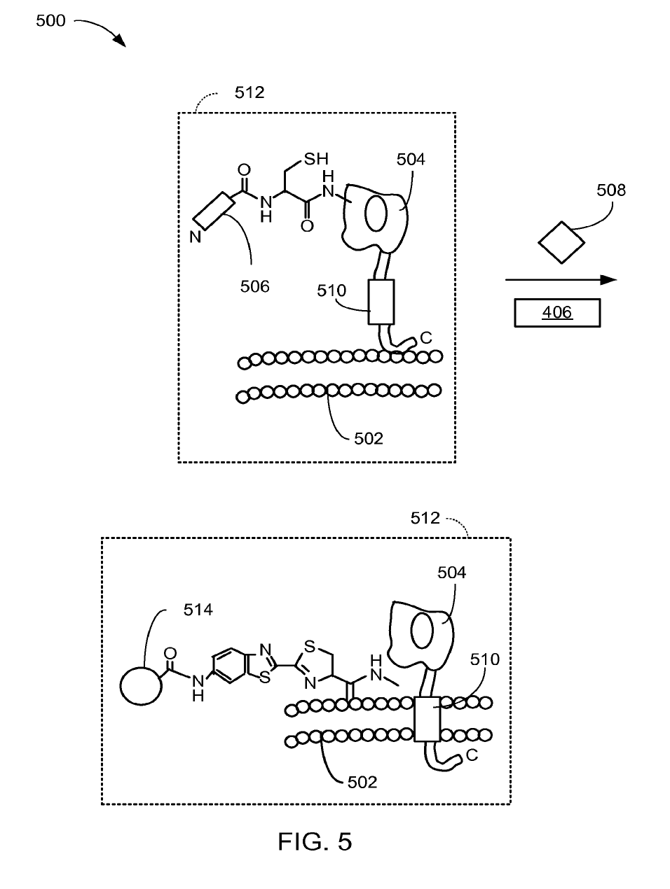

Referring now to FIG. 4, therein is shown a schematic diagram describing a cyclization reaction 400 between a protein 402 with an N-terminal cysteine 404 and a cysteine labeling system 406 consisting of the 2-cyano benzothiazol core 104 with a fluorescent dye 408 covalently linked at 6 position of the 2-cyano benzothiazol core 104. The fluorescent dye 408 may be fluorescein isothiocynate (FITC), rodhamine, cyanine dyes, BODIPY, and ALEXA dyes among many other possible examples.

Referring now to FIG. 5 therein is shown a schematic representation of a labeling strategy 500 for labeling a HeLa cell 502 transfected with a cell cyan fluorescent protein 504 (CFP) having a tobacco etch virus protease substrate 506 fused at its N-terminus. A tobacco etch virus protease 508 is a site-specific enzyme that excises the tobacco etch virus protease substrate 506 leaving a cysteine at the N-terminus (not shown) of the remaining portion of the tobacco etch virus protease substrate 506.

To ensure that the cell cyan fluorescent protein 504 is targeted on the extracellular side of the cell membrane of the HeLa cell 502, a murine Ig κ-chain leader sequence (not shown) was fused to its N-terminus immediately before the tobacco etch virus protease substrate 506. The C-terminus of the cell cyan fluorescent protein 504 was fused with the platelet derived growth factor receptor transmembrane domain 510.. The tobacco etch virus protease treatment will remove the murine Ig κ-chain leader sequence and the N-terminus side of the substrate sequence. The labeling strategy 500 is designed for implementation in a host 512.

The HeLa cell 502 is labeled by co-incubation with the tobacco etch virus protease 508 and the cysteine labeling system 406 with rhodamine 514 acting as the fluorescent dye 408 of FIG. 4. In this embodiment of the present invention, labeling with the cysteine labeling system 406 with rhodamine 514 is a means to confirm transfection of the cell cyan fluorescent protein 504. Following a wash, the fluorescent signal from the rhodamine 514 is only detected after incubation with the tobacco etch virus protease 508, confirming the specificity of the cysteine labeling system 406 to the N-terminus cysteine 510.

In comparison to other chemical labeling approaches, the labeling strategy 500 offers a very small tag with just one amino acid and a simple one-step labeling procedure. In the current form, the labeling can only occur at the N-terminus of the target protein and requires an exogenous enzyme to expose the N-terminal cysteine. However, this limitation may be overcome with the discovery of a peptide motif with a constrained conformation like the FlAsH peptide that can specifically react with CBT. It is also possible to generate the N-terminal cysteine using endogenous proteases in the posttranslational modification such as methinone aminopeptidase.

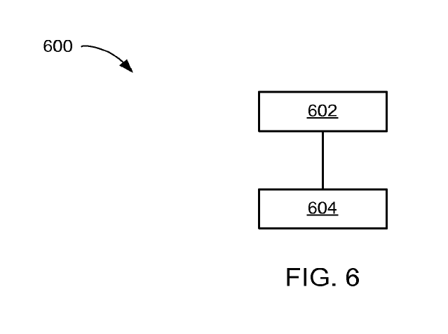

Referring now to FIG. 6, therein is shown a flow chart of a method 600 of use of the activatable bioluminescent probe system 100 in a further embodiment of the present invention. The method 600 includes: providing a 2-cyano benzothial core with a covalently-linked biomolecule X in a reaction environment in a block 602; and reacting the 2-cyano benzothial core to an N-terminal cystenine in a block 604.

The resulting method, process, apparatus, device, product, and/or system is straightforward, cost-effective, uncomplicated, highly versatile, accurate, sensitive, and effective, and can be implemented by adapting known components for ready, efficient, and economical manufacturing, application, and utilization.

Another important aspect of the present invention is that it valuably supports and services the historical trend of reducing costs, simplifying systems, and increasing performance.

These and other valuable aspects of the present invention consequently further the state of the technology to at least the next level.

While the invention has been described in conjunction with a specific best mode, it is to be understood that many alternatives, modifications, and variations will be apparent to those skilled in the art in light of the aforegoing description. Accordingly, it is intended to embrace all such alternatives, modifications, and variations that fall within the scope of the included claims. All matters hithertofore set forth herein or shown in the accompanying drawings are to be interpreted in an illustrative and non-limiting sense.

1. A method of use of a cysteine labeling system comprising:
providing a 2-cyano benzothial core with a covalently-linked biomolecule X in a reaction environment; and
reacting the 2-cyano benzothial core to an N-terminal cystenine.

2. The method as claimed in claim 1 wherein the N-terminal cysteine is part of a peptide.

3. The method as claimed in claim 1 wherein the N-terminal cysteine is part of a protein.

4. The method as claimed in claim 1 wherein the N-terminal cysteine is exposed using a tobacco etch virus protease.

5. The method as claimed in claim 1, wherein the covalently-linked biomolecule X is a fluorescent dye.

6. A cysteine labeling system comprising:
a 2-cyano benzothial core with a covalently-linked biomolecule X in a reaction environment; and
an N-terminal cystenine for forming a bond to the 2-cyano benzothial core.

7. The system as claimed in claim 11 wherein the N-terminal cysteine is part of a peptide.

8. The system as claimed in claim 11 wherein the N-terminal cysteine is part of a protein.

9. The system as claimed in claim 11 further comprising a tobacco etch virus protease for exposing the N-terminal cysteine.

10. The system as claimed in claim 11 wherein the covalently-linked biomolecule X is a fluorescent dye.

11. A method of use of a cysteine labeling system comprising:
providing a 2-cyano benzothial core with a covalently-linked biomolecule X in a host; and
reacting the 2-cyano benzothial core to an N-terminal cystenine through a cyclization reaction between a cyano group in the 2-cyano benzothial core and an amine group and a thiol group in the N-terminal cystenine.

12. The method as claimed in claim 11 wherein the N-terminal cysteine is part of a peptide.

13. The method as claimed in claim 11 wherein the N-terminal cysteine is part of a protein.

14. The method as claimed in claim 11 wherein the N-terminal cysteine is exposed using a tobacco etch virus protease.

15. The method as claimed in claim 11 wherein the covalently-linked biomolecule X is a fluorescent dye.

16. The system as claimed in claim 6 wherein the reaction environment is a host.

17. The system as claimed in claim 16 wherein the N-terminal cysteine is part of a peptide.

18. The system as claimed in claim 16 wherein the N-terminal cysteine is part of a protein.

19. The system as claimed in claim 16 further comprising a tobacco etch virus protease for exposing the N-terminal cysteine.

20. The system as claimed in claim 16 wherein the covalently-linked biomolecule X is a fluorescent dye.
